(19) United States
(12) Patent Application Publication
(10) Pub. No.: US 2017/0189419 A1
(43) Pub. Date: Jul. 6, 2017

(54) PHARMACEUTICAL COMPOSITION FOR TREATING PROSTATE CANCER

(71) Applicant: MA SHEN KAI RUEI CO., LTD., Tainan City (TW)

(72) Inventor: Po-Liang Chen, Taipei City (TW)

(21) Appl. No.: 14/984,605

(22) Filed: Dec. 30, 2015

(51) Int. Cl. A61K 31/575; A61K 31/122

(52) U.S. Cl. CPC A61K 31/575; A61K 31/122

(57) ABSTRACT

The present invention provides a pharmaceutical composition for treating prostate cancer in a subject in need thereof, which comprises a first agent comprising at least a Anctin K (AnK); and a second agent comprising at least an Antroquinonol B (AnQB); as well as each of them can be obtained from the extractions of a fruiting body or a mycelium of Antrodia camphorata wherein the first agent and the second agent shows synergistic effect for use in the treatment of prostate cancer or to prevent or to reduce the risk of a prostate cancer metastasizing, compared to administration of the first agent or the second agent alone.

### BACKGROUND OF THE INVENTION

1. Fields of the Invention

The present invention relates to a pharmaceutical composition for treating prostate cancer in a subject in need thereof, comprising a first agent and a second agent obtained individually from extractions of Antrodia camphorate; especially relates a pharmaceutical composition for treating prostate cancer, which comprises a first agent comprising at least a Anctin K (AnK) and a second agent comprising at least an Antroquinonol B (AnQB).

2. Descriptions of Related Art

The medical term “tumor” refers to the formation of a mass by abnormal cell proliferation which even violates surrounding or distant tissue, affecting the tissue's normal physiological function. The tumors are generally determined to be benign or malignant by histopathological examination. When the healthy cell is taken over, it too can replicate more abnormal cells. The malignant tumors are called cancer. It is known that many types of cancer are caused by genetic aberrations, i.e., mutations. In recent decades, cancer has been one of the top ten causes of death of the people in Taiwan.

Cancer starts when cells begin to grow out of control. Cells in nearly any part of the body can become cancer, and can spread to other areas of the body. A malignant tumor is a group of cancer cells that can grow into (invade) surrounding tissues or spread (metastasize) to distant areas of the body.

The cancer cells grow rapidly and invade surrounding tissue, the walls of nearby blood or lymphatic vessels through bloodstream or lymph system to spread to other parts of the body. The cancer cells stop moving as they are lodged in capillaries at a distant location and divide and migrate into the surrounding tissue, then cancer cells form small tumors at the new location (called micrometastases.)

The lymph system comprises lymph which contains tissue fluid and waste products, as well as immune system cells, lymph nodes which are small, bean-shaped collections of immune system cells being deemed as important in fighting infections, and lymphatic vessels which appear like small veins and are connected with lymph nodes, except that they carry a clear fluid called lymph (instead of blood). Thus, the lymph system is one important way of distribution of cancers to be spread to other parts of the body.

Androgens have been associated with the progression of many diseases such as prostate cancer, benign prostate hypertrophy, male alopecia, acne, and breast cancer, etc. Androgens execute their functions through binding to the androgen receptor (AR) that then regulates gene transcription in the cell nucleus. AR receptor is a member of the nuclear receptor superfamily that acts as a ligand-dependant transcription factor.

AR is expressed mainly in androgen target tissue such as prostate, skeletal muscle, liver, skin and CNS, with the highest expression observed in the prostate. At the cellular level, unbound AR is located mainly in the cytoplasm where it is associated with a complex of heat shock proteins, mainly through interactions with the ligand binding domain (LBD). Upon ligand binding, AR enters the nucleus and activates target genes involved in diverse biological processes such as proliferation, differentiation, apoptosis and secretion. Various AR ligands have been discovered and developed for the treatment of muscle wasting, anemia, benign prostate hyperplasia and prostate cancers (Gao W et al., Drug Discov Today 2007; 12:241-248).

Prostate cancer (PCa) is the most common lethal non-cutaneous malignancy in many Western countries and androgen is one of the major factors influencing PCa development (Hendriksen P J et al., Cancer Res 2006: 66:5012-20). Androgen-AR activity has been tightly associated with the growth, differentiation and even carcinogenesis of the prostate (Culig Z. Urology 2003; 62:21-6; Heinlein C A et al., Endocr Rev 2004; 25:276-308). Furthermore, expression of AR protein is detected in nearly all prostate cancers (PCa) including those of distant metastases and ablation-resistant cases (Shah R B et al., Cancer Res 2004; 64:9209-06).

AR also up-regulates the gene transcription of prostate specific antigen (PSA) which is the most widely used serological biomarker of PCa for both diagnosis and therapeutic assessment (Balk S P et al., J Clin Oncol 2003; 21:383-91; Cleutjens K B et al., Mol Endocrinol 1997; 11:148-61). It is understood that signaling pathways mediated by androgens and AR are essential for both normal and malignant prostate cells, although there are differences between these two states of prostate cells (Hendriksen P J et al., Cancer Res 2006; 66:5012-20; Denmeade S R et al., Prostate 1996; 28:251-65).

At initial diagnosis, most PCa responds to androgen ablation therapy, which prevents the production or blocks the action of androgens, and inhibits prostate cancer growth (Craft N et al., Cancer Res 1999; 59:5030-6). However, cells eventually become androgen-independent or hormone-resistant, and tumors metastasize as the patient's disease advances. This is likely due to the fact that after the development of hormone resistance, many PCa still express AR, which continues to promote PCa progression.

Therefore, targeting AR activity in cancerous prostate cells may be a more effective way for treating prostate cancer as AR is involved at different stages of the disease. In addition, inhibiting AR activity might eventually become beneficial for treating other types of AR-associated diseases such as male alopecia, acne, and breast cancer, etc. Nonetheless, currently existing antiandrogen drugs, such as flutamide, niutamide, and bicalutamide, induce severe side effects and are palliative or ineffective towards terminal prostate cancers Therefore, there is an urgent need to develop or search more effective therapeutic compounds for both prostate cancer and for other diseases associated with AR activity.

The prostate is a male sex gland, about the size of a walnut. It produces a thick fluid that helps propel sperm through the urethra and out of the penisduring sex. Because the prostate is just below the bladder and directly in front of the rectum, a doctor can check the size and condition of the gland by inserting a rubber-gloved finger into the rectum. This digitalrectal exam (DRE) has for years been the gold standard for detecting prostate cancer as well as the noncancerous disorder benign prostatic hyperplasia (BPH).

In 1985, FDA approved the first test for monitoring blood levels of a substance called prostate specific antigen (PSA), which, when elevated, can indicate cancer presence. Several companies now have approved PSA tests, which, experts say, have revolutionized the screening and monitoring of patients.

PSA is an ideal marker for prostate cancer because it is basically restricted to prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

Most PSA tests measure “total PSA,” or the amount that is bound to blood proteins. In March, FDA approved the Tandem R test, which measures not only total PSA but another component called “free PSA,” which floats unbound in the blood. Comparing the two helps doctors rule out cancer in men whose PSA is mildly elevated from other causes. A 1995 study in the Journal of the American Medical Association showed that the free PSA test can reduce unnecessary prostate biopsies by 20 percent in patients with a PSA between 4 and 10.

The availability of increasingly sensitive testing devices has created a debate over when men should be tested for prostate cancer, how often, and whether men under 50 with no symptoms should be routinely screened. Opponents say mass screening would be expensive, and the verdict is still out on whether early detection can curb the disease's mortality rate. But proponents say early detection is the closest thing currently to a cure and that it can save lives. The American Cancer Society and the American Urological Association recommend annual PSA tests—along with the digital exam—for all men over 50 and for high-risk men over 40.

Prostate cancer is the third most common type of cancer in men in the United States. Prostate cancer occurs mainly in older men. About 6 cases in 10 are diagnosed in men aged 65 or older, and it is rare before age 40. The average age at the time of diagnosis is about 66. Still, Prostate cancer is the third most common non-skin cancer in both men and women in Taiwan.

Treatments for cancer can be divided into three categories, namely, surgery, chemotherapy, and radiation therapy. The treatment of surgical resection focuses on removal of the tissue where cancer occurs. However, surgical resection, chemotherapy, and radiation therapy are irreversible methods that will cause destruction of human's cells, tissues, and even organs. Thus, there is an urgent need to provide a method that can effective treat or prevent cancer and will not cause irreversible secondary damage to cancer patients.

Factors that increase the risk of prostate cancer include: older age, a family history of the disease, and race. About 99% of cases occur in those over the age of 50. Having a first degree relative with the disease increases the risk 2 to 3 fold. In the United States it is more common in the African American population than the White American population. Other factors that may be involved include a diet high in processed meat, red meat, or milk products or low in certain vegetables. An association with gonorrhea has been found, but a reason for this relationship has not been identified. Prostate cancer is diagnosed by biopsy. Medical imaging may then be done to determine if the cancer has spread to other parts of the body.

Many cases can be safely followed with active surveillance or watchful waiting. Other treatments may include a combination of surgery, radiation therapy, hormone therapy or chemotherapy. When it only occurs inside the prostate it may be curable. In those in whom the disease has spread to the bones, pain medications, bisphosphonates and targeted therapy, among others, may be useful. Outcomes depend on a person's age and other health problems as well as how aggressive and extensive the cancer is.

Most people with prostate cancer do not end up dying from the disease. The five year survival rate in the United States is 99%. Globally it is the second most common type of cancer and the fifth leading cause of cancer-related death in men. In 2012 it occurred in 1.1 million men and caused 307,000 deaths. It was the most common cancer in males in 84 countries, occurring more commonly in the developed world. Rates have been increasing in the developing world. Detection increased significantly in the 1980s and 1990s in many areas due to increased PSA testing. Studies of males who died from unrelated causes have found prostate cancer in 30% to 70% of those over age 60.

Over time the prostate cancer cells can invade nearby tissues such the underarm lymph nodes or the lungs in a process known as metastasis. The stage of the Prostate cancer, the size of the tumor and its rate of growth are all factors which determine the type of treatment that is offered. Because Prostate cancer is such a major problem of occurrence of relatively aggressive development and existing treatments have limited long-term success, it generally occurs relatively slowly over years and even decades in some cases.

Treatment options include surgery to remove the tumor, drug treatment which includes chemotherapy. Survival rates are often used by doctors as a standard way of discussing a person's prognosis (outlook). Some patients may want to know the survival statistics for people in similar situations, while others may not find the numbers helpful, or may even not want to know them.

Surgery is a common choice to try to cure prostate cancer if it is not thought to have spread outside the gland. The main type of surgery for prostate cancer is known as a radical pro statectomy. In this operation, the surgeon removes the entire prostate gland plus some of the tissue around it, including the seminal vesicles. A radical prostatectomy can be done in different ways.

Survival rates are often based on previous outcomes of large numbers of people who had the disease, but they cannot predict what will happen in any particular person's case. Knowing the type and the stage of a person's cancer is important in estimating their outlook. But many other factors can also affect a person's outlook, such as the grade of the cancer, the genetic changes in the cancer cells, the treatment received, and how well the cancer responds to treatment. Even when taking these other factors into account, survival rates are at best rough estimates. Your doctor can tell you if the numbers below may apply, as he or she is familiar with the aspects of your particular situation.

Although the use of a few therapy has clearly improved the outcome of patients with Prostate cancer, an effective pharmaceutical composition for treating Prostate cancer in a subject in need thereof is still a clinical challenge.

Medicinal plants have been used for centuries to treat a variety of diseases and maintain health before the advent of modern medicine. The accumulation and developing knowledge of the medicinal properties of plants by personal experimentation, local custom, anecdote, and folk tradition leads to the formation of numerous traditional medical systems and therapies, including traditional Chinese medicine (TCM).

Antrodia camphorata and the mycelia products therefrom or thereof possesses edible high value with various excellent functions not only in medicinal such as having anti-oxidant, antihypersensitive and immunostimulatory effects but also the capability of improving physical health by its own anticancer activity, reduced treatment-related symptoms and other side effects similar to medical efficiency of the wild fruiting bodies.

Consequently, many products made from Antrodia camphorata and/or comprising especially the active ingredient extracted form thereof such as Antrodia oil, Antrodia extraction, Antrodia combination and so on, are broadly used in various medicine, health care applications and also Antrodia camphorata and wild cattle camphor thus has been listed as one of the biological treasure in recent years by the Taiwan Government.

Antrodia camphorata is a non-mesh skirt bacteria, an endemic fungus, and grows in the internal heartwood (or the dark/humid wood surface) of Cattle camphorin the mountainous region of Taiwan, altitude 450-2000 meters mountain forest. It is also perennial mushroom fungus and only grows in the inner wall of a wood trunk decayed decades or more, or the lodging of the dead wood wet surface of a Cattle camphor, particularly Cinnamomum kanehirai.

Antrodia camphorata is rich in Triterpenoids, immunostimulatory polysaccharides such as -D-glucan polysaccharides, Adenosine, Nicotinic acid, SOD (superoxide dismutase enzymes), Steroids, Vitamin, essential minerals and other pharmaceutically active principles.

In addition, Antrodia extraction and/or Antrodia oil also contains much important nutrients to the human body, for examples, oleic acid, palmitic acid, linoleic acid, palmitoleic acid, linolenic acid, stearic acid, meat, beans Qu acid, arachidic acid, behenic acid, tetracosanoic acid, n-heptadecyl acid, n-heptadecenoic acid, vitamin A, vitamin B, vitamin E and minerals; as well as it also can inhibit tumor metastasis, reduce the incidence of coronary heart disease, improve immunity and other effects.

Thereby, Antrodia camphorata shows various excellent functions such as detoxification, hypoglycemic effects, reducing blood pressure, improving anti-cancer effect, inhibition of histamine release effect, enhancing anti-inflammatory effect; enhancing immunity, increasing cell viability, eliminating free radicals, promoting liver cell regeneration, lowering down alanine aminotransferase, and in addition to even enhancing the phagocytic capacity of macrophages as well as having the capability in improving physical health.

Although the extracts of Antrodia camphorates, and anticancer agents or compositions comprising thereof, have medical effects as described above and have been attracted attention of people widespread, it still cannot be used as a normal anti-tumor agent for Prostate cancer or used as sole-therapy drug for the treatment of Prostate cancer, due to it is still not clear exactly know what is the particular active ingredient or bioactive composition presented therein.

As to medicine and pharmacy science, there is need of a drug for treatment or amelioration of Prostate cancer with satisfactory effect, there is urgent need to develop a pharmaceutical composition which is capable of solving the defects of anti-Prostate cancer drugs existed in the prior arts and providing excellent medical effects of treating Prostate cancer.

### SUMMARY OF THE INVENTION

In view of defects and problems as above-mentioned, the inventors of the present application conduct several researches with respect to those problems remained in conventional technologies of prior arts.

As results, while an pharmaceutical composition or combination comprising active ingredients or components such as the Anctin K (AnK) and/or Antroquinonol B (AnQB) obtained from the extraction Antrodia camphorates, is used in the treatment and/or prevention of Prostate cancers, it is surprising to find that unexpected excellent effects are achieved as compared to the effects offered by the prior arts of traditional anti-cancer agent.

The unexpected excellent effect include for examples, at least reducing or regulating carcinogenic activity, preventing proliferation or even reversing of cancer cells, and also treating and/or preventing cancer and tumor metastasis.

In addition to those unexpected excellent effects, it also found not only having excellent characteristics chemistry, biology, mechanical science and physical science, but also having good performance of transmittance and transportation.

Further, it is also found that the pharmaceutical composition or combination is very easy for the user uptaking, in short digestion and absorption, and can be used as drugs or adjuvant for the treatment and/or prevention of cancers, especially can inhibit the prevention and treatment of cancer with efficacy while applied to specific cancer cells.

Thus, the present invention is achieved.

Namely, according to an aspect of the present invention, provided is a pharmaceutical composition for treating Prostate cancer in a subject in need thereof, which comprises (1) a first agent comprising at least a Anctin K (AnK) obtained from Extractions of a fruiting body or a mycelium of Antrodia camphorata; (2) a second agent comprising at least an Antroquinonol B (AnQB) obtained from the Extractions of a fruiting body or a mycelium of Antrodia camphorata; wherein the first agent and the second agent shows synergistic effect for use in the treatment of Prostate cancer or to treat, prevent or to reduce the risk of a Prostate cancer metastasizing, compared to administration of the first agent or the second agent alone.

According to another aspect of the present invention, further provided is a pharmaceutical composition as described above, wherein the first agent is a Anctin K (AnK) in a pharmaceutically effective amount for use in the treatment of Prostate cancer or to treat, prevent or to reduce the risk of a Prostate cancer metastasizing; and/or the second agent is an Antroquinonol B (AnQB) in a pharmaceutically effective amount for use in the treatment of Prostate cancer or to treat, prevent or to reduce the risk of a Prostate cancer metastasizing.

Further, according to one aspect of the present invention is to provide a pharmaceutical composition as described above, wherein the first agent and the second agent are in the form of a botanical drug substance (BDS); and may be administered to a human cancer patient in any manner selected from co-administration, a daily regimen, an episodic regimen, intravenous administration, or oral administration.

Furthermore, according to another aspect of the present invention is to provide a pharmaceutical composition as described in claim 1, wherein the Anctin K (AnK) and/or Antroquinonol B (AnQB) is present in an approximate amount of between 80 mg and 90 mg, in the ratio (AnK:AnQB) within the ranges of about 25:35 to about 35:25, about 75:25 to about 25:75, or about 100:17 to about 17:100.

Additionally, according to one aspect of the present invention is to provide a pharmaceutical composition as described above, which further comprises a pharmaceutically acceptable ingredient comprising a vehicle, a carrier, a diluent or an excipient; wherein the excipient comprises an ingredient selected from the group consisting of lactose, sucrose, a mannitol, sorbitol, maize starch, wheat starch, rice starch, potato starch, gelatin and tragacanth.

Besides, according to another one aspect of the present invention is to provide pharmaceutical composition as described above, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

### DETAILED DESCRIPTION OF THE PREFERRED EMBODIMENT

In order to make the spirit and content of the present invention more completely and easily to be understood, various examples of the present invention, particularly various specific embodiments are described in more detailed hereinafter.

However, a skilled person having general knowledge in this technical field pertains to the present invention, shall understand that the present invention is of course not limited to these examples only, and it is possible to achieve the invention by means of taking advantage of other features of function, efficiency or processes which are the same or equal with the present invention.

First, the descriptive instructions or definitions with respect to a term or a description word or phrase particularly used in this specification are separately described below.

Unless defined otherwise, all technical and scientific terms used herein have the same meaning as is commonly understood by one of skill in the art to which the claimed subject matter belongs.

It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

As used herein, a term of “treatment (or treating)” refers to implementing a preventive, curative or palliative disposal for achievement of pharmaceutical and/or physiological effects to an individual subject or a patient having a certain medical condition, symptoms, disease, disorder or an initial condition, in order to partially or completely reduce the severity, delay the occurrence process, and/or inhibit one or more symptoms of the medical condition, abnormal and/or the probability of occurrence of behavior disorders.

As used herein, a term of “effective amount” refers to while a medical drug for cancer is administered (administering, or administration) directly or indirectly in a certain amount, and an effect of reducing the number of cancer cells or particular purpose of treating or preventing a cancer is shown.

The said “certain amount” is so called effective amount.

As used herein, “individual (subject)” or “patient (patient)” can be used interchangeably with one another. The “individual (or individual subject)” or “patient” means, including but not limited to, a human that can accept a compound and/or method for treatment.

Except as otherwise specifically stated that the “individual (or individual subject)” or “patient” may comprise males and females of both sexes. Also, a preferable individual or patient for treatment by using a pharmaceutical composition and/or a method of the present invention is preferably a human.

In this context, the value of the parameter used for defining the scope of the present invention, in essence, inevitably contain standard deviation caused due to individual test methods, and thus it is mostly expressed by an approximate value of number.

However, in particular the implementation of Examples, the value is presented as precisely as possible. In this document, “approximate (or about)” is determined by the skilled person having the usual knowledge of the present invention generally pertains to.

Generally, as used herein, “about” includes an amount that would be expected to be within experimental error. Hence “about 10 μg” means “about 10 μg” and also “10 μg”. It also refers to the actual value which falls in the range of an acceptable standard deviation including the exact amount, for example, the actual value is expressed by a ±10%, it means within a range, ±5%, ±1%, or ±0.5% of a particular value.

According to the present invention, a pharmaceutical composition for treating Prostate cancer in a subject in need thereof is provided. The pharmaceutical composition comprises a first agent and a second agent obtained individually from extractions of Antrodia camphorate.

According to the present invention, the first agent used in the pharmaceutical composition for treating Prostate cancer, may comprise, but are not limited to at least a Anctin K (AnK). In this context, the “dehydroeburicoic acid (DeHBA)” generally has a chemical structure usually represented by Formula (2) as shown below, with molecular formula of C₃1H₄₈O₃ and molecular weight of 468.7.

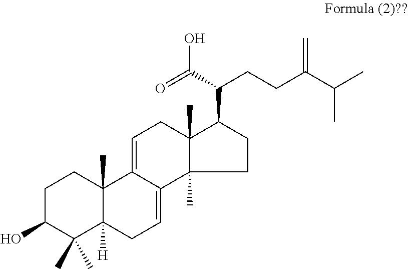

According to the present invention, Anctin K (AnK) used as the first agent in the pharmaceutical composition for treating Prostate cancer may be obtained by purification or isolation from the Extractions of Antrodia camphorata, which is rich in Anctin K (AnK) and another bioactive ingredients for cancer treatment.

It is reported that the extractions extracted from the Antrodia camphorata comprise many effective bio-ingredients in cancer treatment, for example, Sesquiterpenoids (sesquiterpene compounds), Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like.

In the extractions extracted from the Antrodia camphorata, the Sesquiterpenoids that can be used as a cancer-treatment-effective bio-ingredient may comprise, but are not limited to, for example Antrocin and the like.

Further, in the extractions extracted from the Antrodia camphorata, the Diterpenoids (diterpene compounds) that can be used as a cancer-treatment-effective bio-ingredient may comprise, but are not limited to, for example 19-hydroxylabda-8(17)-en-16,15-olide, 3β,19-dihydroxylabda-8(17),11E-dien-16,15-olide, 13-epi-3β,19-dihydroxyl-abda-8(17),11E-dien-16,15-olide, 19-hydroxylabda-8(17),13-dien-6,15-olide,14-deoxy-11, 12-didehydroandrograph-olide, 14-deoxy-andrographolide, pinusolidic acid and so on.

Furthermore, in the extractions extracted from the Antrodia camphorata, the Triterpenoids that can be used as a cancer-treatment-effective bio-ingredient may comprise, but are not limited to, for example Camphoratin B, camphoratin A, Antcin K, Antcin I (zhankuic acid B, 3α-hydroxy-4α-methylergost-8,24(28)-dien-7,11-dione-26-oic acid), camphoratin E, antcin H (zhankuicacid C, 3α,12α-dihydroxy-4α-methylergost-8,24(28)-dien-7,11-dione-26-oic acid), methyl antcinate H (3α,12α-dihydroxy-7,11-dioxo-4α-methylergost-8,24(28)-dien-26-oate), zhankuic acid E, camphoratin C, camphoratin H, camphoratin I, antcin A (1,4α-methylergost-8,4(8)-diene-3,11-dion-26-oic acid), camphoratin J, methyl antcinate A (methyl 4α-methylergost-8,24(28)-dien-3,11-dion-26-oate), antcin E (3,11-dioxo-4α-methylergost-8,14,24(28)-trien-26-oic acid), antcin C (7β-hydroxy-4α-methylergost-8,24(28)-diene-3, 11-dion-26-oic acid), camphoratin G, antcin F (3,11-dioxo-7β-hydroxy-4α-methylergost-8,14,24(28)-trien-26-oic acid), camphoratin D, camphoratin F, methyl antcinate G (7 α-acetoxy-3,11-dioxo-4α-methylergost-8,24(28)-dien-26-oate), antcin B (zhankuicacid A, 4α-methylergost-8,24(28)-dien-3,7,11-trion-26-oic acid), antcin D (zhankuicacid F, 14-hydroxy-4α-methyl-3,7,11-trioxoergost-8,24(28)-dien-26-oic acid), methyl antcinate B (methyl 4α-methylergost-8,24(28)-dien-3,7,11-trion-26-oate), zhankuic acid D, eburicol (24-methylenedihydrolanosterol), eburicoic acid (35),7 sulphurenic acid, versisponic acid D, dehydroeburicoic acid, dehydrosulphurenic acid, 15α-acetyldehydrosulphurenic acid, 3β,15α-dihydroxylanosta-7,9(11),24-triene-21 oicacid, epi-friedelinol and so on. Steroids generally comprise β-Sitosterol, stigmasterol (44),16 ergosterol peroxide, ergosterol D, ergosterol, β-sitostenone, ergosta-4,7,8(14),22-tetraen-3-one, ergosta-2,4,8(14),22-tetraen-3-one an so on.

Additionally, in the extractions extracted from the Antrodia camphorata, the Furan ring structures such as five (Furan) class or pyrazolyl (Pyrrole) class that can be used as a cancer-treatment-effective bio-ingredient may comprise, but are not limited to, for example Antrocinnamomin C (3-isobutyl-4-(4-hydroxyphenyl)furan-2,5-dione), 3-isobutyl-4-[4-(3-methyl-2-butenyloxy)phenyl]furan-2,5-dione antrocinnamomin D (2-hydroxy-3-isobutyl-4-[4-(3-methylbut-enyloxy) phenyl]-2H-furan-5-one), cis-3-(4-hydroxyphenyl)-4-isobutyl-dihydrofuran-2,5-dione, dimethyl-2-(4-hydroxyphenyl)-3-isobutyl-maleate, 3-(4-hydroxyphenyl)-4-isobutyl-1H-pyrrole-2,5-dione, 3-iso-utyl-4-[4-(3-methyl-2-butenyloxy) phenyl]-1Hpyrrole-2,5-dione (antrodin B, camphorataimide B), trans-3-isobutyl-4-[4-(3-methyl-2-butenyloxy)phenyl]pyrrolidine-2,5-dione, antrocinnamomin B (3-isobutyl-4-(4-hydroxyphenyl)-1H-pyrrol-1-ole-2,5-dione), 3-isobutyl-4-[4-(3-methyl-2-butenyloxy)phenyl]-1H-pyrrol-1-ol-2,5-dione (antrodin C, camphorataimide C), antrocinnamomin A (3-isobutyl-4-[4-(3-methyl-2-butenyloxy)phenyl]-1H-pyrrol-1-acetoxyl-2,5-dione), trans-1-hydroxy-3-(4-hydroxyphenyl)-4-isobutylpyrrolidine-2,5-dione, 3R,4S-1-hydroxy-3-isobutyl-4-[4-(3-methyl-2-butenyloxy) phenyl]pyrrolidine-2,5-dione), antrodin D (camphorataimide D, 3R, 4R-1-hydroxy-3-isobutyl-4-[4-(3-methyl-2-butenyloxy)phenyl]pyrrolidine-2,5-dione), antrodi-oxolanone and so on.

Besides, in the extractions extracted from the Antrodia camphorata, the Lignoids that can be used as a cancer-treatment-effective bio-ingredient may comprise, but are not limited to, for example (+)-sesamin, (−)-sesamin, 4-hydroxysesamin, Aptosimon and so on.

In the extractions extracted from the Antrodia camphorata, the Benzenoids that can be used as a cancer-treatment-effective bio-ingredient may comprise, but are not limited to, for example 1,4-dimethoxy-2,3-methylene-dioxy-5-methylbenzene, methyl 2,5-di-ethoxy-3,4-methylene-dioxybenzoate, 4,5-dimethoxy-2,3-methylene-dioxybenzoic acid, 2,4,5-trimethoxybenzaldehyde, 2,3-methylene-dioxy-6-methylbenzene-1,4-diol, 2,4-dimethoxy-6-methylbenzene-1,3-diol, benzocamphorin C, 5-methylbenzo[1,3]-dioxole-4,7-dione, 2-methoxy-5-methyl[1,4]benzo-quinone, 2,3-dimethoxy-5-methyl[1,4] benzoquinone, isobutylphenol, 2,3,4,5-tetramethoxybenzoylchloride, 2,2,5,5,-tetra-methoxy-3,4,3,4,-bis(methylenedioxy)-6,6,-dimethylbiphenyl, benzocamphorin E, benzocamphorin D, antrocamphin A, antrocamphin B, benzocamphorin A, benzocamphorin B and so on.

More specifically, in the extractions extracted from the Antrodia camphorata, the other compounds that can be used as a cancer-treatment-effective bio-ingredient may comprise, but are not limited to, for example α-Tocospiro B, methyloleate, antroquinonol, antroquinonol B, 4-acetylantroquinonol B, adenosine, cordycepin and so on.

In some embodiments, the first agent may comprise sub-ingredient which is other than the main component of Anctin K (AnK) and for example, may be one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient of the first agent may comprise, but are not limited to at least one selected from the group consisting of antroquinonol, antrocinnamonin A, antrocinnamonin B, antroquinonol D, zhankuic acid A, zhankuic acid C, antcin K, antcin C, and a mixture thereof.

According to the present invention, the second agent used in the pharmaceutical composition for treating Prostate cancer, may comprise, but are not limited to at least Antroquinonol B (AnQB). In this context, the “Antroquinonol B (AnQB)” generally has a chemical structure usually represented by Formula (1) as shown below, with molecular formula of C₂₄H₃₈O₅ and molecular weight of 406.

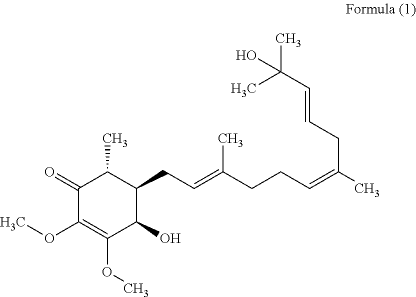 [SMILES: COC1=C(OC)[C@H](O)[C@H](C/C=C(\C)CC/C=C(/C)C/C=C/C(C)(C)O)[C@@H](C)C1=O]

In some embodiments, the second agent may comprise Antroquinonol B (AnQB) as a main ingredient and a sub-ingredient such one compound selected form Sesquiterpenoids, Diterpenoids, Triterpenoids, Steroids, furan ring structure such as five member (Furan) or pyrazolyl class (Pyrrole), lignan compound (Lignoids), benzene compounds (Benzenoids), superoxide dismutase and amino acids and the like. Preferably, the sub-ingredient may comprise, but are not limited to at least one selected from the group consisting of antroquinonol, antrocinnamonin A, antroquinonol D, dehydrosulphurenic acid, zhankuic acid A, zhankuic acid C, antcin K, antcin C, and a mixture thereof.

According to the present invention, the extractions used for obtaining the first agent and/or the second agent of the pharmaceutical composition of the present invention, is not particularly limited, for example, can be isolated from the fruiting body or mycelium of Antrodia camphorata by using a conventional purification method well known in prior arts.

Whether raw materials are the fruiting body or mycelium of Antrodia camphorata or not, an extraction method suitable for use in general, includes non-polar solvent extraction, highly polar solvent extraction, low polar solvent extraction, high temperature extraction, lower temperature extraction method, the combination of their supercritical extraction method or the like.

For example, a solvent suitable used for extracting Antrodia camphorata typically includes water, inorganic solvents, organic solvents and the like. The organic solvents used in the present invention may include, but not limited to, alcohols such as methanol, ethanol or propanol, esters such as ethyl acetate, alkanes such as hexane, or halogenated alkanes such as chloromethane, chloroethane. Among them, water and ethanol are preferred, and ethanol is particularly preferred.

Further, extraction temperature suitably used for extracting Antrodia camphorata in the present invention is generally not particularly limited, for example, it can be conducted at below 0° C., further it also may be conducted in the lower temperature range of 0° C. to 40° C., or at a higher temperature range of above 50° C. to 150° C.

In illustrative embodiments, the Antroquinonol B (AnQB) and Anctin K (AnK) may be obtained by isolation and/or purification processes from the extractions of Antrodia camphorata. The isolation and/or purification processes used in the present invention may include, but not limited to, liquid chromatography, gas chromatography, gas-liquid chromatography, high-performance liquid chromatography (HPLC) and the likes.

Particularly, the Extraction A of Antrodia camphorata containing the first agent and/or the second agent used in the pharmaceutical composition of the present invention, was preferably obtained by using a specific extracting method comprising steps of (A) extracting fruiting bodies of Antrodia camphorata with hot water at a temperature in a range of 45° C.˜100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.˜15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCF.

According to the present invention, the effects such as treating Prostate cancer, inhibiting the growth of Prostate cancer cell and others, of Antroquinonol B (AnQB) and/or Anctin K (AnK) may be tested by using any of the test methods used in the prior arts, for example, MTT assay of using 3-(4, 5-dimethylthiazol-2-yl)-2, S-diphenyl tetrazolium bromide (MTT) to determine cell survival rates of Prostate cancer cell lines.

In some embodiments, through MTT assays, it is proved that Antroquinonol B (AnQB) and/or Anctin K (AnK) can decrease the survival rates of prostate cancer cell lines (PC3 and DU-145) at the same time and half-maximal inhibitory concentration (IC50) values comes to be relatively low.

Thus, the pharmaceutical composition comprising a first agent of at least a Anctin K (AnK) and a second agent of at least a Antroquinonol B (AnQB) of the present invention is very useful in inhibiting growth of preferably Prostate cancer cell. Also, the pharmaceutical composition of the present invention can thus be further used in preparation of a medicinal composition for treating Prostate cancer, which has improved the therapeutic effects with respect to the prior arts.

Further, according to one aspect of the present invention, first agent and the second agent of the pharmaceutical composition as described above, may be made to be in the form of but not limited to a botanical drug substance (BDS); and may be administered to a human cancer patient in any of manners such as the one selected from co-administration, a daily regimen, an episodic regimen, intravenous administration, or oral administration.

According to one aspect of the present invention, the effective amount of Anctin K (AnK) of the first agent and Antroquinonol B (AnQB) of the second agent existed in the pharmaceutical composition as described above, may be an pharmaceutical amount useful for treating of Prostate cancer, or preventing or reducing the risk of a Prostate cancer metastasizing.

according to some embodiments of the present invention, the effective amount of Anctin K (AnK) of the first agent and Antroquinonol B (AnQB) of the second agent may individually be but not limited to 0.01 mg˜2000.0 mg. For example, it is suitable to be administrated within the range of 0.01 mg˜10.0 mg, preferably within the range of 0.01 mg˜8.50 mg, more preferably within the range of 0.01 mg˜6.50 mg, particularly preferably within the range of 0.01 mg˜5.00 mg.

Furthermore, according to one aspect of the present invention, the ratio of Anctin K (AnK) and Antroquinonol B (AnQB) in the pharmaceutical composition as described above, shown as (ZhAD:AnQB) may be but not limited to within the ranges of about 1.0:1.0 to about 1.0:20.0. For example, it is suitable to be administrated within the range of about 1.0:1.0 to about 1.0:15.0, preferably within the range of about 1.0:1.0 to about 1.0:9.0, more preferably within the range of about 3.0:1.0 to about 1:9.0, particularly preferably within the range of about 9.0:1.0 to about 1.0:9.0.

Additionally, according to another one aspect of the present invention, the pharmaceutical composition may further comprises a pharmaceutically acceptable ingredient a vehicle, a carrier, a diluent or an excipient. For example, the excipient suitable used in the present invention is an ingredient, a compound or a component selected from the group consisting of lactose, sucrose, a mannitol, sorbitol, maize starch, wheat starch, rice starch, potato starch, gelatin and tragacanth, or composition or combination thereof.

Besides, according to another one aspect of the present invention, the pharmaceutical composition may further comprise an additive. For example, the additive suitable used in the present invention is at least an ingredient, a compound or a component selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, solubilizers, wetting agents, and a mixture thereof.

In some embodiments, the pharmaceutical compositions of the present invention may be formed in a liquid formulation which is suitable for use in oral administration, for example, oral suspensions, emulsions, micro-emulsions, and/or curing liquid (elixirs). In the case of the liquid formulation, the active ingredients of the pharmaceutical compositions of the present invention may be further blended with various formulations, for example, sweetening or flavoring agents, coloring matter or dyes, if necessary, it may be further blended with emulsifying and/or suspending agents, or such as water, alcohol, propylene glycol, glycerin and other diluents, or maintain buffer pH values.

Besides, in other embodiments, the formulations comprising a liquid pharmaceutical composition of the present invention may be prepared into sterile injectable solutions or suspensions; for example, it may be manufactured into a solution that is suitable for intravenous injection, intramuscular injection, it in intraperitoneal injection, or subcutaneous administration, and others.

Diluents suitable for use in a sterile injectable solution or suspension described above, for example, may include, but are not limited to, 1,3-butanediol, mannitol, water, Ringer's solution, isotonic chloride sodium; optionally natural oils or fatty acids acceptable in pharmacy, such as oleic acid, glycerol derivatives, or such as olive oil or canola oil, and the like.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### EXAMPLES

The details of the examples for the present invention are described as follows.

### Example 1 Extraction a Obtained from Antrodia camphorata

Firstly, an Extraction A was obtained by extracting fruiting bodies of Antrodia camphorata by using a specific method comprising steps of (A) extracting fruiting bodies of Antrodia camphorata with hot water at a temperature in a range of 45° C.˜100° C. to obtain Extractions HW; (B) extracting the residues HW by a fractional distillation to obtain Extractions FD which are collected from a condensation liquid in a fractional distillation apparatus; (C) extracting the residues FD by immersing with a low polar solvent at least for 4 hours to obtain Extractions LPS; (D) extracting the residues LPS through a cryo-condensation process by dropping a iced ethanol/water with a temperature in a range of 0° C.˜15° C. to obtain Extractions IEW; (E) extracting the residues IEW through a SCF (supercritical fluid extraction) by using CO₂ as a solvent at a temperature of 31.26° C. and a pressure of 72 atm to obtain Extractions SCF.

Subsequently, Extractions HW, Extractions FD, Extractions LPS, Extractions IEW and Extractions SCF were mixed uniformly to form a mixture denoted as Extraction A. In the Extraction A

### Example 2 Ex Vivo Survival Assay for Anti-pPC3 and DU-145 Prostate Cancer Effects

The Antroquinonol B (AnQB) and Anctin K (AnK) separately isolated from the Extraction A of Example 1 were added into the culture media of human prostate-cancer cells, PC3 or DU-145, to test for tumor cell survival by using anti-cancer drug screen model. This survival assay was carried out with the widely known MTT (3-[4,5-dimethylthiazol-2-yl]2,5-diphenyltetrazolium bromide) assay.

PC3 (PC-3) human prostate cancer cell lines are one of the cell lines used in prostate cancer research. These cells are useful in investigating the biochemical changes in advanced prostatic cancer cells and in assessing their response to chemotherapeutic agents. Moreover, they can be used to create subcutaneous tumors in mice in order to investigate a model of the tumor environment in the context of the organism.

PC3 have low testosterone-5-alpha reductase and acidic phosphatase activity,[3] do not express PSA (prostate-specific antigen), and are PSMA-negative (prostate-specific membrane antigen). Furthermore, karyotypic analysis has shown that PC3 are near-triploid, presenting 62 chromosomes. Q-band analysis showed no Y chromosome. From a morphological point of view, electron microscopy revealed that PC3 show characteristics of poorly-differentiated adenocarcinoma. They have features common to neoplastic cells of epithelial origins, such as numerous microvilli, junctional complexes, abnormal nuclei and nucleoli, abnormal mitochondria, annulate lamellae, and lipoidal bodies.

DU145 (DU-145) and PC3 human prostate cancer cell lines are the “classical” cell lines of prostatic cancer. DU145 cells have moderate metastatic potential compared to PC3 cells which have high metastatic potential.

The DU145 cell line was derived from brain metastasis.[3] DU145 are not hormone-sensitive and do not express prostate-specific antigen (PSA).

It has been demonstrated that administration of NF-kappaB ligand RANKL promoted DU145 cell invasion in bone, resulting in osteolytic lesions. DU145 cells also produce soluble factors that activate pre-osteoblast precursors and increase RANKL expression, thus facilitating prostate cancer metastasis in bone.

The human prostate cancer cell lines, PC-3 and DU-145 were cultivated in media containing fetal calf serum for 18 hours. The proliferated cells were washed once with PBS, then treated with 1× trypsin-EDTA and centrifuged at 2500 rpm for 80 minutes. The supernatant was discarded and the cell pellet was re-suspended in 200 ml of fresh culture medium by gently shaking. The cells were placed in a 96-well plate.

Subsequently, the plates were read on an ELISA reader to determine the survival rates. The half maximal inhibitory concentration (IC50) values were calculated and results of ex vivo survival assay were thus obtained.

According to those results, it shows that the Prostate cancer cells are capable of decreasing the survival rate of prostate cancer cell PC-3 and DU-145 and namely, it is concluded that the Antroquinonol B (AnQB) and Anctin K (AnK) can inhibit the growth of Prostate cancer cell. On the other hand, in a pharmaceutically effective amount of the Antroquinonol B (AnQB) and Anctin K (AnK) can be applied to the treatment of Prostate cancer, or to prevent or to reduce the risk of a Prostate cancer metastasizing.

### Example 3˜8 Pharmaceutical Compositions of the Invention

The active ingredient of a first agent consisting of Anctin K (AnK) and a second agents consisting of Antroquinonol B (AnQB) separated from an Extraction A of Antrodia camphorates of Example 1, was uniformly mixed according to the composition ratio shown in table 1 to be formulated as the pharmaceutical compositions of the present invention used for a prevention and/or treatment of a Prostate cancer.

TABLE 1
The Pharmaceutical Compositions of The Invention

| | | First Agent | Second Agents |
|---|---|---|---|
| | | (AnK) | (AnQB) |
| Example 3 | Composition A (wt. %) | 75 | 25 |
| Example 4 | Composition B (wt. %) | 85 | 15 |
| Example 5 | Composition C (wt. %) | 55 | 45 |
| Example 6 | Composition D (wt. %) | 95 | 5 |
| Example 7 | Composition E (wt. %) | 65 | 35 |
| Example 8 | Composition F (wt. %) | 55 | 45 |

AnK: Anctin K
AnQB: Antroquinonol B

By using the same manner as the method described above, respectively human Prostate cancer cell line PC-3 and DU-145 was cultured. After then, the pharmaceutical compositions A˜F of the present invention was applied separately, and then MTT assay for each sample was measured to assess the inhibition effectiveness of various cancer cell activity while using pharmaceutical compositions A˜F of the present invention.

According to those test results of cancer suppression activity, it confirmed that human Prostate cancer cell line PC-3 and DU-145 can be suppressed by the pharmaceutical compositions A˜F of the present invention, particularly all of IC50 (μg/ml) are significantly better than the prior art. In summary, the results show that the pharmaceutical composition of the present invention is a potentially useful drug having pharmaceutical effectiveness in the treatment of various cancers.

What is claimed is:

1. A pharmaceutical composition for treating Prostate cancer in a subject in need thereof, which comprises
(1) a first agent comprising at least a Anctin K (AnK) obtained from extractions of a fruiting body or a mycelium of Antrodia camphorata;
(2) a second agent comprising at least an Antroquinonol B (AnQB) obtained from the extractions of a fruiting body or a mycelium of Antrodia camphorata;
wherein the first agent and the second agent shows synergistic effect for use in the treatment of prostate cancer or to prevent or to reduce the risk of a prostate cancer metastasizing, compared to administration of the first agent or the second agent alone.

2. The pharmaceutical composition as described in claim 1, wherein the first agent is a Anctin K (AnK) in a pharmaceutically effective amount for use in the treatment of Prostate cancer, or to prevent or to reduce the risk of a Prostate cancer metastasizing.

3. The pharmaceutical composition as described in claim 1, wherein the second agent is an Antroquinonol B (AnQB) in a pharmaceutically effective amount for use in the treatment of Prostate cancer, or to prevent or to reduce the risk of a Prostate cancer metastasizing.

4. The pharmaceutical composition as described in claim 1, wherein the the first agent and the second agent are in the form of a botanical drug substance (BDS).

5. The pharmaceutical composition as described in claim 1, wherein the Anctin K (AnK) and/or Antroquinonol B (AnQB) is present in an approximate amount of between 80 mg and 90 mg.

6. The pharmaceutical composition as described in claim 1, wherein the ratio of Anctin K (AnK) to Antroquinonol B (AnQB) is between about 25:35 to about 35:25 (AnK:AnQB).

7. The pharmaceutical composition as described in claim 1, wherein the ratio of Anctin K (AnK) to Antroquinonol B (AnQB) is between about 75:25 to about 25:75 ((AnK:AnQB).

8. The pharmaceutical composition as described in claim 1, wherein the ratio of Anctin K (AnK) to Antroquinonol B (AnQB) is between about 100:17 to about 17:100 (AnK:AnQB).

9. The pharmaceutical composition as described in claim 1, which further comprises a pharmaceutically acceptable ingredient comprising a vehicle, a carrier, a diluent or an excipient.

10. The pharmaceutical composition as described in claim 1, wherein the first agent and the second agent are administered to treat with cancer patient.

11. The pharmaceutical composition as described in claim 1, wherein the first agent and the second agent are co-administered.

12. The pharmaceutical composition as described in claim 1, wherein the first agent and the second agent are administered in a daily regimen.

13. The pharmaceutical composition as described in claim 1, wherein the first agent and the second agent compositions are administered in an episodic regimen, intravenous administration, or oral administration.

14. The pharmaceutical composition as described in claim 1, wherein the excipient comprises an ingredient selected from the group consisting of lactose, sucrose, a mannitol, sorbitol, maize starch, wheat starch, rice starch, potato starch, gelatin and tragacanth.

15. The pharmaceutical composition as described in claim 1, wherein further comprises at least one additive selected from the group consisting of absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, bactericides, sweeteners, wetting agents, and a mixture thereof.

16. The pharmaceutical composition as described in claim 1, wherein the Prostate cancer cells are selected from the group consisting of: prostate cancer cell line PC-3; and prostate cancer cell line DU-145.
